(19) United States
(12) Patent Application Publication
(10) Pub. No.: US 2016/0317480 A1
(43) Pub. Date: Nov. 3, 2016

(54) Composition and Method of Pain Relief, Reduction of Inflammation, Maintenance of Joints, and Soothing Muscle Soreness

(71) Applicant: Nicholas Adam Brockman, Hawesville, KY (US)

(72) Inventor: Nicholas Adam Brockman, Hawesville, KY (US)

(21) Appl. No.: 14/698,595

(22) Filed: Apr. 28, 2015

(51) Int. Cl. A61K 31/167; A61K 31/125; A61K 36/28; A61K 31/7008; A61K 31/726; A61K 31/10; A61K 36/886; A61K 31/045

(52) U.S. Cl. CPC A61K 31/167; A61K 36/886; A61K 31/125; A61K 31/045; A61K 31/7008; A61K 31/726; A61K 31/10; A61K 36/28

(57) ABSTRACT

A Composition and Method of Pain Relief, Reduction of Inflammation, Maintenance of Joints, and Soothing Muscle Soreness which alleviates pain and inflammation from muscle strain, spasms, arthritis, and bursitis. The composition and method for alleviation of pain and inflammation generally includes a combination of Lidocaine HCL, camphor, menthol, Bisabolol, glucosamine hydrochloride (glucosamine HCI), chondroitin, and MSM, along with a number of other ingredients. The composition described and claimed herein has been shown to alleviate pain and inflammation from injuries and conditions such as muscle strains, spasms, arthritis, and bursitis. The composition will preferably be incorporated into a topical cream which may be applied to the skin around the affected area to provide not only immediate relief from pain and inflammation, but also long-lasting relief which may last for days or even weeks.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a composition and more specifically it relates to a Composition and Method of Pain Relief, Reduction of Inflammation, Maintenance of Joints, and Soothing Muscle Soreness which alleviates pain and inflammation from muscle strain, spasms, arthritis, and bursitis.

2. Description of the Related Art

Any discussion of the related art throughout the specification should in no way be considered as an admission that such related art is widely known or forms part of common general knowledge in the field.

There is a growing need for topical creams for the treatment of pain and inflammation from injuries such as muscle strain, spasms, arthritis, and bursitis. As one gets older, it is increasingly common for joints to become inflamed, such as due to arthritis. Sports and exercise injuries are also increasingly common in a society that is growing more and more health conscious over the years. Although oral pharmaceuticals are shown to be effective for treatment of these conditions, they often carry undesirable side effects such as addiction or organ (such as liver) problems.

Because of the inherent problems with the related art, there is a need for a new and improved Composition and Method of Pain Relief, Reduction of Inflammation, Maintenance of Joints, and Soothing Muscle Soreness which alleviates pain and inflammation from muscle strain, spasms, arthritis, and bursitis.

### BRIEF SUMMARY OF THE INVENTION

The invention generally relates to a composition which includes a combination of lidocaine HCL, camphor, menthol, Bisabobol, glucosamine hydrochloride (glucosamine HCI), chondroitin, MSM, and a number of other ingredients. The composition described and claimed herein has been show to alleviate pain and inflammation from injuries and conditions such as muscle strains, spasms, arthritis, and bursitis. The composition will preferably be incorporated into a topical cream which may be applied to the skin around the affected area to provide not only immediate relief from pain and inflammation, but also long-lasting relief which may last for days or even weeks.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### DETAILED DESCRIPTION OF THE INVENTION

### A. Overview.

The present invention is directed to a Composition and Method of Pain Relief, Reduction of Inflammation, Maintenance of Joints, and Soothing Muscle Soreness. More specifically, the composition described and claimed herein has been shown to alleviate pain and inflammation from injuries and conditions such as muscle strains, spasms, arthritis, and bursitis. The composition will preferably be incorporated into a topical cream which may be applied to the skin around the affected area to provide not only immediate relief from pain and inflammation, but also long-lasting relief which may last for days or even weeks. It should be appreciated that the present invention may be incorporated into a range of mediums, and need not necessarily be comprised of a topical cream as described herein. It should also be appreciated that the components or ingredients of the composition may vary for different applications or embodiments of the present invention.

### B. Composition.

i. Primary Ingredients.

The present invention is comprised of a composition which may be topically applied to the body to alleviate pain and inflammation. The composition's primary active ingredients include lidocaine HCL, camphor, menthol, Bisabolol, glucosamine hydrochloride (glucosamine HCI), chondroitin, and MSM. These primary active ingredients, when combined in a composition such as a topical cream, will act to treat both acute or chronic pain and inflammations; particularly those associated with muscle strain, spasms, arthritis, and/or bursitis.

a. Lidocaine HCI is a drug which is generally applied topically to stop itching and pain from various skin conditions (such as eczema, bums, etc.) and to treat minor discomfort and itching caused by inflammations such as hemorrhoids. Lidocaine HCI is generally prepared in a cream, lotion, spray, foam or gel for topical application to the skin.

b. Camphor is readily absorbed through the skin producing either a coolness or warmth sensation by activating the ion channel TRPV3, and acts as slight local anesthetic and antimicrobial substance

c. Menthol may also be included in the composition as a primary ingredient for its analgesic properties. As a topical analgesic, it is used to relieve minor aches and pains, such as muscle cramps, sprains, headaches and similar conditions, alone or combined with chemicals such as camphor, eucalyptus oil or capsaicin

d. Bisabolol [(-)-alpha-bisabolol)], also known as Alpha Bisabolol or levomenol, is the primary constituent of German Chamomile (Matricaria chamomilla or Matricaria recultita), and is also found naturally in the bark of the Candela (Vanillosmopsis erythropappa) tree of Brazil, which accounts for most of the world's production of this ingredient. It is a terpene and is classed as an alcohol. This product is naturally derived from plant material and it is the (-) a Bisabolol isomer, the active principle in the plant.

a-Bisabolol is a naturally occurring active ingredient that accelerates the healing process of skin and protects it from the effects of daily stress. Known to have anti-irritant and anti-inflammatory properties, a-bisabolol has been shown to reduce UV-induced erythema in-vivo. It has also been demonstrated in studies to enhance the dermal penetration of other ingredients, making it a truly versatile active ingredient for skin care products, especially those containing other active ingredients and cosmeceuticals. It also has natural anti-bacterial as well as anti-mycotic properties.

e. Glucosamine hydrochloride (glucosamine HCI) is a pharmaceutical which has been shown to act as a joint degeneration retardant Glucosamine HCI may thus be included as a secondary ingredient in the present invention to improve the composition's joint degeneration treatment properties.

f. Chondroitin is in dietary supplements used as an alternative medicine to treat osteoarthritis and also approved and regulated as a symptomatic slow-acting drug for this disease.

g. Methyl Sulfonyl Methane (MSM) is sold as a dietary supplement and marketed with a variety of claims, often in combination with glucosamine and/or chondroitin for helping to treat or prevent osteoarthritis.

### II. Secondary Ingredients.

The composition described herein will also include one or more secondary ingredients which may be included in the composition in combination with the primary ingredients. By way of example and without limitation, the secondary ingredients may include such components as aloe vera, flower extract such as from heterotheca inuloides, C12-15 aklyl benzoate, coconut oil and glycerin, ceteareth-20, dimethicone, emu oil, glycerine, glyceryl stearate (GMS), polyethylene glycol (PEG) such as PEG-100 stearate, phenoxyethanol, ethylhexylglycerin, polysorbate 80, propylene glycol, sodium hyaluronate (HLA), and/or water as described herein.

The present invention may include aloe vera within the composition. Aloe vera is well known as a plant species which soothes inflammations such as bums, moisturizes the skin and aids in healing minor wounds. An aloe vera gel will be included as part of the secondary ingredients of the composition claimed herein to improve the soothing effects of the composition. In some embodiments, aloe vera may be omitted.

The present invention may also include extract from flowers of the heterotheca inuloides flower (sometimes referred to as amica inuloides). Heterotheca inuloides has been shown to have an anti-inflammatory and analgesic effect and has been used as a traditional medicine in Mexico. Extract from the flowers of the heterotheca inuloides may be included as part of the secondary ingredients of the composition claimed herein to improve the anti-inflammatory and analgesic effects of the composition.

The present invention may also include alkyl benzoate, commonly comprised of a benzoic acid and long-chain alcohols. Alkyl benzoate has been shown to be effective both as an antimicrobial and skin conditioning agent. Preferably, C12-15 alkyl benzoate will be included as part of the secondary ingredients of the composition claimed herein to improve its antimicrobial and skin conditioning properties.

The present invention may also include coconut oil, such as caprylic triglyceride oil 28 or capric triglyceride oil. Glycerin may also be utilized. These products are commonly utilized as an emollient which has skin-repairing properties. They are commonly used in various products both as a thickener as well as to improve the moisturization and replenishment provided by the products. Coconut oils and/or glycerin may be included as part of the secondary ingredients of the composition claimed herein as a thickening agent and emollient to Improve moisturization and replenishment of skin around the affected area.

The present invention may also include cetostearyl alcohol or, more specifically, ceteareth-20. Ceteareth-20 is a mixture of fatty alcohols such as cetyl and staryl alcohols. Ceteareth-20 has been shown to be an emulsion stabilizer, opacifying agent, foam boosting surfactant, viscosity increasing agent, and emollient. Ceteareth-20 may be included

Dimethicone, which is a mixture of polydimethylsiloxanes and silicon dioxide, is sometimes referred to as simethicone. Dimethicone is included in the a preferred embodiment of the presently described composition to act as an antifoaming agent and carminative, as well as to provide skin protection

Emu oil, comprised of fat from an emu, may be included as a secondary ingredient in the composition. Emu oil has been shown to act as an emollient which heals skin.

Glyceryl stearate, sometimes referred to as glycerol monostearate or GMS, is an organic molecule used as an emulsifier. In a preferred embodiment of the present 6 invention, glyceryl stearate SE may be included as an emulsifying agent.

Polyethylene glycol, sometimes referred to as PEG, is a polyether compound which acts as a surfactant and increases lubrication. PEG may thus be included in the present invention to act as a surfactant and lubricant for the composition.

Phenoxyethanol is glycol ether commonly utilized in skin creams as a bactericide and preservative. Ethyl hexylglycerin is a deodorizing agent Phenoxyethanol and ethyl 23 hexylglycerin may thus be included as secondary ingredients of the composition to aid in preservation and prevent formation of bacteria over time and to act as a deodorizer for the composition.

Polysorbates may also be included in the composition as an emulsifier.

Propylene glycol is an organic compound which acts as a humectant and viscosity decreasing agent, a coupling agent, and a solvent, may be included in the composition.

Hyaluronan, which is sometimes referred to as sodium hyaluronate or hyaluronic acid, is an anionic nonsulfated glycosaminoglycan which has been shown to have anti-aging effects and acts as a water binding agent to fill spaces between connective fibers such as collagen and elastin in the dermis.

The composition may also include some quantity of water.

### C. Method of Use of Preferred Embodiment.

The present composition will preferably be adapted for topical application to the body. Preferably, the present composition will be comprised of a cream. The cream may be applied to portions of the body which are afflicted by pain or inflammation, such as area of skin adjacent to inflamed joints or injuries (such as bums or skin damage). The combination of primary ingredients will act to alleviate pain and inflammation in the areas adjacent to application of the cream. The secondary ingredients will improve various qualities of the cream such as its viscosity, analgesic effects, and preservation over time.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. Composition and Method of Pain Relief, Reduction of Inflammation, Maintenance of Joints, and Soothing Muscle Soreness comprising primary ingredients:
Lidocaine HCL, camphor, menthol, Bisabolol, glucosamine hydrochloride (glucosamine HCI), chondroitin, and MSM
And comprising secondary ingredients:
Aloe vera, flower extract such as from heterotheca inuloides, C12-15 aklyl benzoate, coconut oil and glycerin, ceteareth-20, dimethicone, emu oil, glycerine, glyceryl stearate (GMS), polyethylene glycol (PEG) such as PEG-100 stearate, phenoxyethanol, ethylhexylglycerin, polysorbate 80, propylene glycol, sodium hyaluronate (HLA), and/or water.
